(19) United States
(12) Patent Application Publication
(10) Pub. No.: US 2023/0207110 A1
(43) Pub. Date: Jun. 29, 2023

(54) SYSTEMS, DEVICES, AND METHODS FOR OPTIMIZING MEDICAL PROCEDURE ERGONOMICS

(71) Applicant: MAKO Surgical Corporation, Fort Lauderdale, FL (US)

(72) Inventors: Laura SCHOLL, Sparta, NJ (US); Emily HAMPP, Far Hills, NJ (US); Antonia F. CHEN, Newton, MA (US)

(73) Assignee: MAKO Surgical Corporation, Fort Lauderdale, FL (US)

(21) Appl. No.: 18/145,504

(22) Filed: Dec. 22, 2022

Related U.S. Application Data
(60) Provisional application No. 63/265,973, filed on Dec. 23, 2021.
(60) Provisional application No. 63/364,061, filed on May 3, 2022.

(51) Int. Cl. G16H 40/20; G06Q 10/0635

(52) U.S. Cl. CPC G16H 40/20; G06Q 10/0635

(57) ABSTRACT

A computer-implemented method for optimizing medical procedure ergonomics may include receive baseline ergonomic data from a database, determine one or more ergonomic safe zones based on the receive baseline ergonomic data, receive ergonomic measurements from sensors within a medical suite and sensors attached to a body of a medical staff, determine current medical staff ergonomics based on the received baseline ergonomic data and received ergonomic measurements during a medical procedure, determine whether the determined current medical staff ergonomics exceed any of the determined one or more ergonomic safe zones, and upon determining that the determined current medical staff ergonomics exceed any of the determined one or more ergonomic safe zones, providing ergonomic feedback to the medical staff.

### CROSS-REFERENCE TO RELATED APPLICATION(S

This patent application claims the benefit of priority to U.S. Provisional Patent Application Nos. 63/265,973, filed Dec. 23, 2021, and 63/364,061, filed on May 3, 2022, the entirety of which is incorporated herein by reference.

### TECHNICAL FIELD

Various embodiments of the present disclosure relate generally to providing guidance during medical procedures and, more particularly, to evaluating medical personnel ergonomics during a medical procedure and providing alerts and feedback to the medical personnel.

### BACKGROUND

Some medical procedures, such as orthopedic surgery, may be demanding on the musculoskeletal health of the personnel performing the procedure, such as the surgeon and other operating room staff. For example, 44-66% of orthopedic surgeons reported work-related discomfort or other musculoskeletal issues attributed to poor surgeon posture. (See, for example, “What factors influence surgeon cervical posture and perceived workload during TKA?,” L. Scholl et al., EPiC Series in Health Sciences, Volume 3, 2019, pp. 331-333, citing. Alqahtani et al., JoA 31 (2016), pp. 1194-1198.) Additionally, hospital nurses routinely take on ergonomically challenging tasks, with soft tissue retraction listed as one of the highest risks. Thus, there is a desire to reduce the risk of enduring such discomforts or other musculoskeletal issues that can arise during medical procedures.

The present disclosure is directed to overcoming one or more of these above-referenced challenges.

### SUMMARY OF THE DISCLOSURE

According to certain aspects of the present disclosure, systems and methods are disclosed for optimizing medical procedure ergonomics.

In one embodiment, a computer-implemented method is disclosed for optimizing medical procedure ergonomics, the method comprising: receive baseline ergonomic data from a database, determine one or more ergonomic safe zones based on the receive baseline ergonomic data, receive ergonomic measurements from sensors within a medical suite and sensors attached to a body of a medical staff, determine current medical staff ergonomics based on the received baseline ergonomic data and received ergonomic measurements during a medical procedure, determine whether the determined current medical staff ergonomics exceed any of the determined one or more ergonomic safe zones, and upon determining that the determined current medical staff ergonomics exceed any of the determined one or more ergonomic safe zones, providing ergonomic feedback to the medical staff.

In accordance with another embodiment, a system is disclosed for optimizing medical procedure ergonomics, the system comprising: a data storage device storing instructions for optimizing medical procedure ergonomics in an electronic storage medium; and a processor configured to execute the instructions to perform a method including: receive baseline ergonomic data from a database, determine one or more ergonomic safe zones based on the receive baseline ergonomic data, receive ergonomic measurements from sensors within a medical suite and sensors attached to a body of a medical staff, determine current medical staff ergonomics based on the received baseline ergonomic data and received ergonomic measurements during a medical procedure, determine whether the determined current medical staff ergonomics exceed any of the determined one or more ergonomic safe zones, and upon determining that the determined current medical staff ergonomics exceed any of the determined one or more ergonomic safe zones, providing ergonomic feedback to the medical staff.

In accordance with another embodiment, a non-transitory machine-readable medium storing instructions that, when executed by the a computing system, causes the computing system to perform a method for optimizing medical procedure ergonomics, the method including: receive baseline ergonomic data from a database, determine one or more ergonomic safe zones based on the receive baseline ergonomic data, receive ergonomic measurements from sensors within a medical suite and sensors attached to a body of a medical staff, determine current medical staff ergonomics based on the received baseline ergonomic data and received ergonomic measurements during a medical procedure, determine whether the determined current medical staff ergonomics exceed any of the determined one or more ergonomic safe zones, and upon determining that the determined current medical staff ergonomics exceed any of the determined one or more ergonomic safe zones, providing ergonomic feedback to the medical staff.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, and serve to explain the principles of the disclosed embodiments.

FIG. 1 depicts an exemplary medical procedure scenario, according to one or more embodiments.

FIG. 2 depicts a system for optimizing medical procedure ergonomics, according to an exemplary embodiment.

FIG. 3 depicts sensors and other input information related to optimizing medical procedure ergonomics, according to an exemplary embodiment.

FIG. 4 depicts a flowchart of a method of optimizing medical procedure ergonomics, according to one or more embodiments.

FIG. 5 depicts output of a method of optimizing medical procedure ergonomics, according to an exemplary embodiment.

FIG. 6 depicts a flowchart of a method of optimizing medical procedure ergonomics, according to one or more embodiments.

### DETAILED DESCRIPTION OF EMBODIMENTS

Various embodiments of the present disclosure relate generally to evaluating medical personnel ergonomics during a medical procedure and providing alerts and feedback to the medical personnel.

The terminology used below may be interpreted in its broadest reasonable manner, even though it is being used in conjunction with a detailed description of certain specific examples of this disclosure. Indeed, certain terms may even be emphasized below; however, any terminology intended to be interpreted in any restricted manner will be overtly and specifically defined as such in this Detailed Description section.

As discussed above, some medical procedures, such as orthopedic surgery, may be demanding on the musculoskeletal health of the personnel performing the procedure, such as the surgeon and other operating room staff, leading to increased reported work-related discomfort attributed to poor posture and ergonomically challenging tasks. However, such issues are not limited to a surgical procedure in an operating room, but may occur with any other medical procedures.

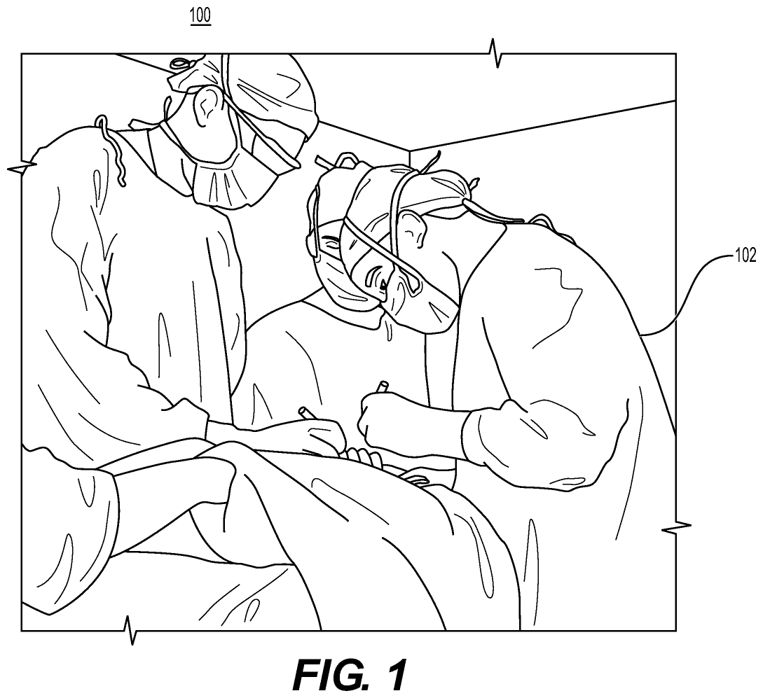

FIG. 1 depicts a typical scenario 100 in which a medical staff 102 performs a medical procedure on a patient. Medical staff 102 may be, for example, a surgeon, nurse, technician, assistant, anesthesiologist, radiologist, trainee, or other medical personnel. During the course of the procedure, medical staff 102 may be required to stand for extended periods, potentially in awkward postures, to gain access to the patient, and may also be required to pull or put pressure on instruments for extended periods. Such extended exertions may lead to poor ergonomics for the medical personnel, possibly resulting in musculoskeletal injuries.

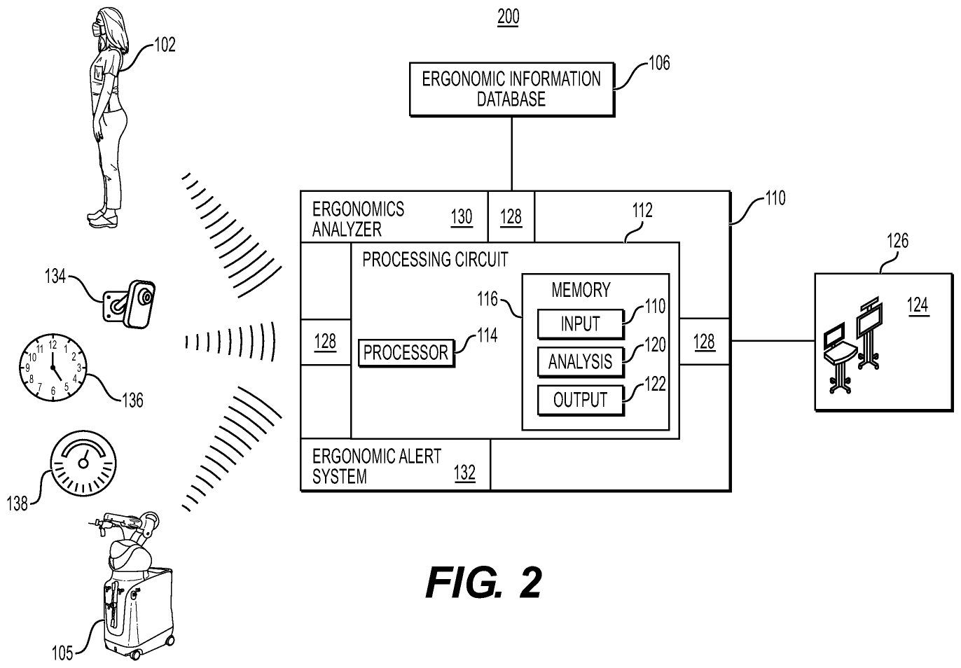

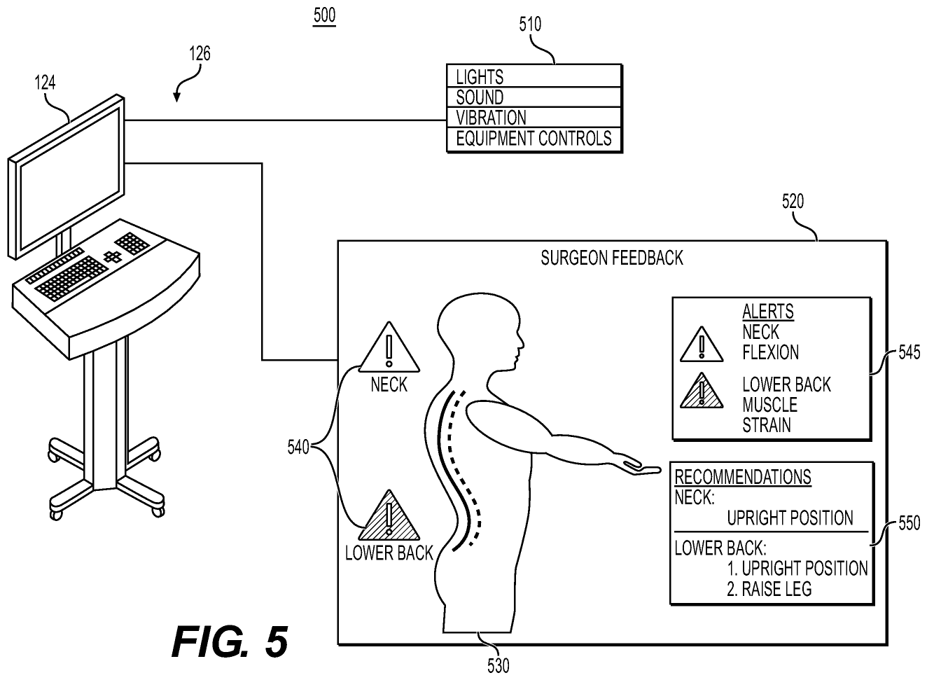

FIGS. 2-5 and the discussion that follows describe systems and methods to monitor and analyze the ergonomics of medical personnel during a medical procedure, and provide assistance, such as, for example, alerts, feedback, and/or adjustments to medical equipment to mitigate poor ergonomics and possibly prevent or reduce resulting musculoskeletal injuries.

### Exemplary Embodiment

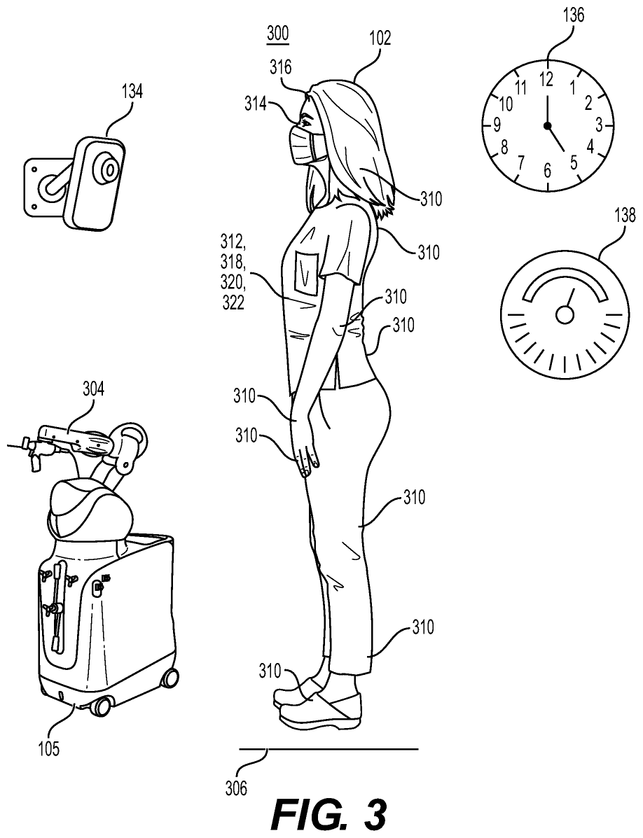

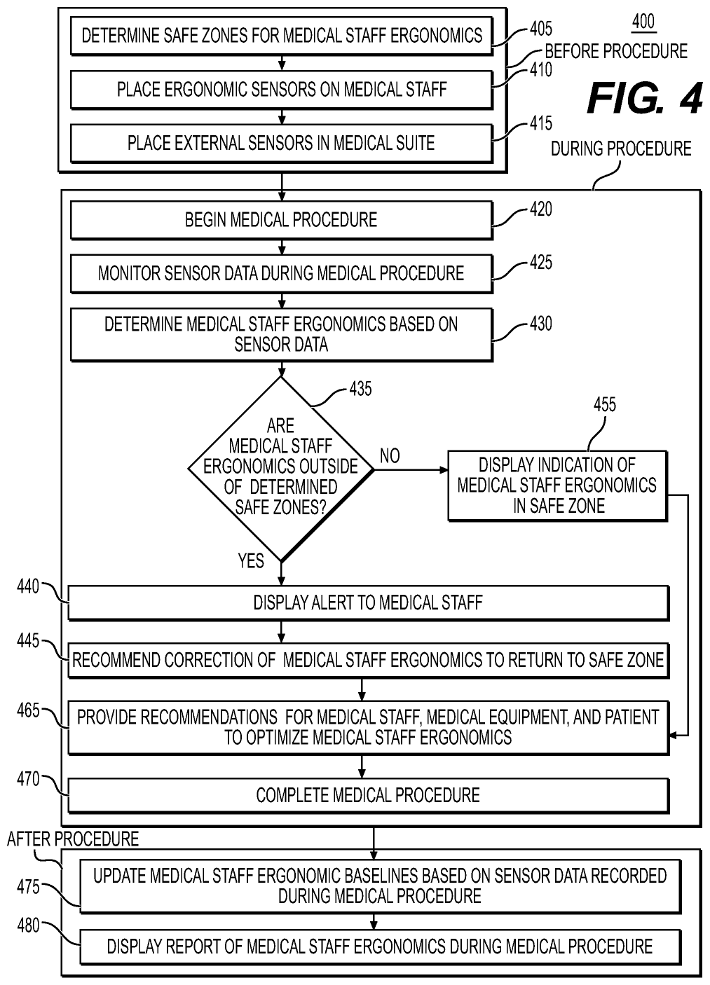

FIG. 2 illustrates a system 200 for optimizing medical procedure ergonomics, FIG. 3 illustrates exemplary sensors for measuring ergonomics during a medical procedure, and FIG. 4 illustrates a method for optimizing medical procedure ergonomics. The system 200 may include an ergonomics optimizer 110 configured to analyze ergonomic data obtained during a medical procedure with respect to a database 106 of ergonomic information, and to provide ergonomic alerts and feedback 126 to medical staff 102 performing the medical procedure.

Database 106 of ergonomic information may include demographic information of medical staff 102 performing the medical procedure and/or the patient undergoing the medical procedure. Demographic information may include, for example, height, weight, body mass index (BMI), age, gender, prior injuries, and pregnancy. Demographic information may further include general ergonomic limits and personal ergonomic limits of medical staff 102. Ergonomic limits may be specific to procedures similar to the medical procedure being performed or may be generalized for multiple medical procedures.

Database 106 of ergonomic information may also include anthropometric data for medical staff 102. Anthropometric data may, for example, represent segment or limb length information for medical staff 102, technicians, support staff, and patients, and may be gathered, for example, by direct measurement or may be estimated based on image capture either during or outside of a medical procedure. The segment or limb length information may include, for example, dimensional measurements, such as, for example, length, circumference, maximum and minimum joint angles, spacing, etc., for various body segments and limbs of medical staff 102, technicians, support staff, and patients, the body segments and limbs including, for example, toes, feet, lower leg, upper leg (thigh), fingers, hands, forearm, upper arm (bicep), hips, spine, shoulders, neck, head, jaw, nose, eyes, ears, etc. Anthropometric data generated from image capture or other indirect means may be referred to as human pose estimation. Such anthropometric and kinematic data may be collected from medical staff 102 and may be used to optimize ergonomic configurations of the medical suite workspace, as discussed in detail below. For example, in general, ergonomic configurations of the medical suite workspace may be defined based on object type, position, and size. Such object parameters may include, for example, the selection and placement of instruments, including robotic systems 105 and their attachments such as various end effectors, the placement, size, and height of various tables, including instrument tables and patient tables, and the placement, size, and height of various displays and interactive terminals, etc. These object parameters may be based on the feedback from the anthropometric data and used to enhance interaction of medical staff 102 with objects and instruments within the medical suite as well as other medical staff 102, technicians, support staff, and patients within the medical suite. These ergonomic recommendations would support ergonomic positions throughout medical procedure in order to reduce risk of fatigue and injury and ergonomic/kinematic demands on medical staff 102. Additionally, different instruments, such as, for example, end-effectors for robotic systems 105 could be included to support medical staff 102 ergonomics based on the anthropometric data. The anthropometric data may suggest medical suite efficiencies which include but are not limited to medical suite setup and organization as well as potential tool or instrument selection. Anthropometric data may be used to provide recommendations for modular attachments for instruments, robots, tables, etc. that could support an improved position or posture of medical staff 102. Anthropometric data may be gathered from medical staff 102, technicians, support staff, and patients within the medical suite, and may be captured, for example, from motion capture cameras or other devices or may be directly measured with analog devices, such as devices worn by medical staff 102.

As an example of provision of different instruments based on biomentric, anthropometric, and ergonomic data for medical staff 102, technicians, support staff, and patients, ergonomics optimizer 110 may recommend particular instruments and end effectors for particular medical procedures. For example, ergonomics optimizer 110 may recommend use of an offset impactor and reamer handle to access the acetabulum depending on the patient's anthropometrics.

Database 106 of ergonomic information may also include biometric data for medical staff 102. Biometric data may, for example, be used to estimate physiological functions. Use of biometric sensors, such as is discussed below, may provide the ability to collect an assortment of data related to physical performance and capacities of medical staff 102, technicians, support staff. Biometrics such as, for example, activity, heartrate, stress, sleep, etc., may be used to assess activities of medical staff 102, technicians, and support staff inside the medical suite during a medical procedure, as well as outside the medical suite during normal daily activities during the minutes, hours, or days prior to the medical procedure. For example, physical exertion of medical staff 102, technicians, and support staff may be tracked such as through various fitness trackers, smart watches, etc., as may periods or instances of elevated or abnormal heartrate or other cardiac function, duration and depth of periods of sleep, etc. In addition, metabolic rates, blood glucose, hydration, water and calorie intake, and/or other nutrition information such as intake of sugar, carbohydrates, protein, vitamins, etc. may be measured or gathered. Metabolic rates, including a basal metabolic rate (BMR) and a resting metabolic rate (RMR), may be measured, such as through calorimetry, or may be estimated using any known estimating equation based on, for example, age, gender, weight, height, calorie intake, and fat-free mass, etc. Blood glucose may be measured, for example, through use of test strips or continuous glucose monitors (CGM). Hydration may be measured based on urine color, urinalysis, blood analysis, salivary analysis, or recoded impressions such as thirst level. Water and calorie intake may be estimated based on individual recorded intake, such as a nutrition and hydration diary. Other nutrition information may be estimated based on individual recorded intake, such as a nutrition and hydration diary, or may be measured, such as through urinalysis, blood analysis, salivary analysis. Biometric data may, for example, be used to assess features such as cardiovascular demand, physical demand, and even recovery. Biometric data may further be used to assess overall demand of a medical procedure through estimates of heart rate of medical staff 102, technicians, and support staff and combinations of other measured biometric data, anthropometric and kinematic data, and other data. The data can be used enhance medical procedure efficiency and scheduling based on medical staff 102 performance and physiological state. For example, smart watches, chest straps, and even mobile platforms may be used to collect biometric data and measure physiological parameters, such as, for example, periods of physical exertion, periods or instances of elevated or abnormal heartrate or other cardiac function, duration and depth of periods of sleep, etc. Such biometric data may complement the robot-assisted medical procedures and enhance interactions between medical staff 102 and robotic systems 105. Further, biometric data, anthropometric and kinematic data, and other data may be used to generate scores or assessments of medical staff 102, technicians, and support staff to support scheduling medical procedures and enhance interactions between medical staff 102 and robotic systems 105. Biometric data may be gathered from medical staff 102, technicians, support staff, and patients within the medical suite, and may be captured, for example, from motion capture cameras or other devices or may be directly measured with analog devices, such as devices worn by medical staff 102.

Database 106 of ergonomic information may also include other information about past medical procedures. For example, for each prior medical procedure there may be a designation of the difficulty of the procedure. This designation may be based on subjective opinions provided by medical staff 102, technicians and support staff, such as a questionnaire or post-procedure survey of medical staff 102, technicians and support staff. For example, medical staff 102, technicians and support staff may rate the difficulty of a medical procedure on a subjective scale such as, for example, “easy,” “medium,” and “hard” with possible gradations between, numeric scales, such as “1-10,” “0-100,” etc. The assessment of medical staff 102, technicians and support staff may further be estimated based on, for example, a semantic analysis of statements made by medical staff 102, technicians and support staff following the medical procedures, such as, for example, in spoken comments, text messages, emails, written reports, etc. such semantic analysis may be based, for example, on the use of certain words or phrases expressing opinions about the difficulty of the medical procedure, such as, for example, “tough,” “a breeze,” “not too bad,” etc., as well as various exclamations, such as, for example, “whew,” “ouch,” “yay,” etc. Alternatively, or in addition, this designation may be based on an objective analysis of attributes of the procedure, including, for example, the type of procedure, the biometrics and anthropometrics of medical staff 102, technicians, support staff, and patients, the time spent on the procedure as a whole and/or the time spent on a sub-procedure of the procedure, measurements of forces placed on instruments used in the procedure, measurements of forces applied by robotic systems 105, the clinical outcome of the procedure, etc. Patient demographics that may be employed in such analysis may include, for example, patient height, weight, muscle strength, bone mineral density, age, type of disease, etc. The information about past medical procedures in database 106 may include time index information, possibly allowing for a correlation among the recorded data.

Database 106 of ergonomic information may also include other information about past medical procedures. For example if one of medical staff 102, technicians and support staff changes the conditions of a medical procedure, such as, for example, changing settings or configuration of robotic systems 105, changing the height or position of equipment within the medical suite, such as, for example, instrument table tray placement and size; patient table height and placement, positions of any of medical staff 102, technicians and support staff, etc., such changes may be recorded in database 106, possibly including a time index to allow a correlation of the changes with associated biometric measurements of medical staff 102, technicians, support staff, and the patient. Such data and calculated correlations may be used, for example, in evaluating the difficulty of a medical procedure, as discussed in detail elsewhere in this description. The changes made during the medical procedure may be presented to medical staff 102, technicians and support staff following the medical procedure for confirmation and for inclusion in future medical procedures.

The procedure difficulty data in database 106 may be used to create a model of medical procedures that may later be used to, for example, predict a difficulty level of a procedure. Such a model may, for example, correlate reported difficulty level with procedure type, changes to the conditions of the medical procedure made by one of medical staff 102, technicians and support staff during the procedure, demographic, anthropometric, and biometric data of medical staff 102, technicians, support staff, and patients, etc. and may individualize such predictions for each individual among medical staff 102, technicians, and support staff. The model may further recognize and account for trends in reported difficulty levels of procedures for individuals among medical staff 102, technicians, and support staff. That is, if an individual reports decreasing difficulty levels for a particular procedure, the model may produce lower predictions of difficulty levels of similar future procedures. For example, a particular medical procedure that may have been predicted as “difficult” previously may later be predicted as “medium” or “moderate” based on the individual's reports of decreasing difficulty levels for similar medical procedures. Similar changes may be reflected in other subjective scales of procedure difficulty, such as are discussed above.

Procedure difficulty level data and procedure outcome data may further be used to assess a level of expertise for an individual among medical staff 102, technicians, and support staff with respect to a particular procedure. For example, if an individual consistently assesses a procedure as “difficult,” or as having greater difficulty that other procedures, or as having higher difficulty than the assessments of other individuals for similar medical procedures, that individual may be determined to have a lower level of expertise for that procedure. Conversely, an individual assessing procedures as less difficult may be determined to have a higher level of expertise for those procedures. For example, an individual's expertise may be assessed on a scale such as, for example, “novice” or “intern,” “medium” or “resident,” and “expert” or “attending,” with possible gradations between, or on a numeric scale, such as “1-10,” “0-100,” etc.

The ergonomics optimizer 110 may make other recommendations for medical procedures. For example, ergonomics optimizer 110 may recommend a particular placement of robotic systems 105 or a particular configuration of robotic systems 105, such as left- or right-handed, selection of tool types, such as straight or offset, selection of tool lengths, etc. In addition, ergonomics optimizer 110 may recommend positions of medical staff 102, technicians, and support staff, and of various equipment and fixtures in the medical suite. The recommended layout of the medical suite may include recommended placement of cameras and other anatomy tracking devices in the medical suites, such as to reduce collisions with the trackers and prevent issues with line of sight requirements of the trackers. The recommended layout and configuration of the medical suite may be reinforced through images provided on a display and through auditory or visual feedback to correct or verify the positioning of medical staff 102, technicians, and support staff, and of the various equipment and fixtures in the medical suite. Such recommendations may be based on information about past medical procedures stored in database 106, including, for example, biometric data, anthropometric data, changes made to medical suite ergonomics or workflow, procedure outcomes, confirmed changes or preferences made by medical staff 102, technicians, and support staff, and any other data described elsewhere in this description. In addition, ergonomics optimizer 110 may show a picture on screen of where any of medical staff 102, technicians, and/or support staff should stand, based on, for example, biometric, demographic and ergonomic information about any of the individuals, the location of robotic systems 105, the location of the camera, etc. This may make robot registration less fatiguing for the individual, and easier for the individual to achieve proper registration.

The ergonomics optimizer 110 may receive input ergonomic data prior to, during, and after the medical procedure from multiple sensors, such as sensors incorporated in robotic systems 105, various sensors 134, 136, and 138 in the medical suite, medical staff 102 and associated sensors, or other components of a medical procedure. Details of the various sensors and the generated data will be discussed in detail below. In this disclosure, “user” is synonymous with “practitioner” or “medical personnel” and may be any person completing the described action (e.g., surgeon, technician, nurse, etc.).

Ergonomics optimizer 110 may provide ergonomic alerts and feedback 126 to medical staff 102 performing the medical procedure by way of one or more information interfaces 124 that may be positioned within the medical suite. Details of ergonomic alerts and feedback 126 and the one or more information interfaces 124 will be discussed below.

Ergonomics optimizer 110 may also provide analysis and recommendations with respect to scheduling of procedures performed by medical staff 102, technicians, or support staff. For example, such scheduling may be determined in order to reduce physical stress or fatigue of medical staff 102, technicians, or support staff based on an analysis of the difficulty level of each medical procedure according to procedure difficulty data stored in database 106. For example, ergonomics optimizer 110 may recommend that an individual among medical staff 102, technicians, or support staff be limited to a single high-difficulty medical procedure per day, or that a sequence of medical procedures assigned to such an individual meet certain requirements, such as, for example, not presenting two high-difficulty medical procedures sequentially. In addition, ergonomics optimizer 110 may take into account a predicted difficulty level for a planned procedure for each individual among medical staff 102, technicians, or support staff and recommend assigning the procedure to individuals with lower predicted difficulty levels.

### Ergonomics Optimizer

The ergonomics optimizer 110 may be utilized to implement the various functions (e.g., calculations, processes, analyses) described herein. Ergonomics optimizer 110 may include a processing circuit 112 having a processor 114 and memory 116. Processor 114 can be implemented as a general purpose processor, an application specific integrated circuit (ASIC), one or more field programmable gate arrays (FPGAs), a group of processing components, or other suitable electronic processing components. Memory 116 (e.g., memory, memory unit, storage device, etc.) may be one or more devices (e.g., RAM, ROM, Flash-memory, hard disk storage, etc.) for storing data and/or computer code for completing or facilitating the various processes described in the present application. Memory 116 may be or include volatile memory or non-volatile memory. Memory 116 may include database components, object code components, script components, or any other type of information structure for supporting the various activities described in the present application. According to an exemplary embodiment, memory 116 may be communicably connected to processor 114 and may include computer code for executing one or more processes described herein. The memory 116 may contain a variety of modules, each capable of storing data and/or computer code related to specific types of functions. In one embodiment, memory 116 contains several modules related to medical procedures, such as an input module 118, an analysis module 120, and an output module 122.

Ergonomics optimizer 110 may comprise ergonomics analyzer 130 and ergonomics alert system 132. The operation of these components will be described in detail below.

It should be understood that the ergonomics optimizer 110 need not be contained in a single housing. Rather, components of ergonomics optimizer 110 may be located in various locations of the system 200 depicted in FIG. 2, or even in a remote location. For example, ergonomics analyzer 130 and ergonomics alert system 132 may be components of a single module or may be embodied as separate modules.

The present disclosure contemplates methods, systems, and program products on any machine-readable media for accomplishing various operations. The machine-readable media may be part of or may interface with the procedure optimizer 110. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage, other magnetic storage devices, solid state storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Referring again to FIG. 2, the ergonomics optimizer 110 further includes one or more communication interfaces 128. The communication interfaces 128 can be or include wired or wireless interfaces (e.g., jacks, antennas, transmitters, receivers, transceivers, wire terminals, etc.) for conducting data communications with external sources via a direct connection or a network connection (e.g., an Internet connection, a LAN, WAN, or WLAN connection, etc.). For example, communication interfaces 128 can include an Ethernet card and port for sending and receiving data via an Ethernet-based communications link or network. In another example, communication interfaces 128 can include a Wi-Fi transceiver for communication via a wireless communications network. Thus, if the procedure optimizer 110 is physically separate from other components of the system 200 shown in FIG. 2, such as the robotic systems 105, the various sensors 134, 136, and 138, medical staff 102 and associated sensors, or other components of a medical procedure, the communication interfaces 128 can enable wireless communications between the ergonomics optimizer 110 and these separate components.

### Ergonomics Sensors

As discussed above, ergonomics optimizer 110 may receive input ergonomic data during the medical procedure from multiple sensors placed within the medical suite. FIGS. 2 and 3 depict a variety of such sensors and other input information, according to an exemplary embodiment. As shown in FIG. 3, provided sensors worn by or attached to medical staff 102 may include, for example, a variety of motion wearable sensors 310, eye motion glasses 314, head mounted display 316, and additional biometric sensors 312, 318, 320, and 322. Medical provider may stand on a pressure sensor 306, such as, for example, a foot pedal. In addition, sensors placed within the medical suite may include camera 134, clock or timer 136, and thermometer and/or humidity sensor 138. Robotic surgical instruments 105 may include sensors 304 to measure robotic arm location and motion. Although a single instance of each sensor may be depicted in FIGS. 2 and 3, the embodiments are not so limited and multiple instances of each sensor may be provided. Likewise, multiple medical staff 102, each with their own worn or attached sensors 310-322 may be present and participating in the medical procedure. Finally, although not depicted in FIGS. 2 and 3, additional sensors related to the position and motion of a patient, similar to the motion wearable sensors 310 and additional biometric sensors 312, 318, 320, and 322 attached to and/or worn by medical staff 102.

Motion wearable sensors 310 may monitor, for example, medical staff 102 steps, range of motion of one or more medical staff 102 body segments (i.e. knee, hip, lower back, shoulder, wrist, neck), weight bearing time of medical staff 102, activity time of medical staff 102, and the gait of medical staff 102. Motion wearable sensors 310 may further monitor limb position of medical staff 102 such as range of motion, flexion, current angles (e.g. joint angles, other anatomical angles), peak angle, time spent at the current or peak angles, or another particular angle, and muscle activity and/or strain for the head-neck, mid-back, lower back, pelvis/hip, shoulder, elbow, wrist, finger joints, knee, ankle and foot, etc., of medical staff 102. Motion wearable sensors 310 may further monitor pressures and loads exerted at the fingertips, feet, and toes of medical staff 102. Motion wearable sensors 310 may include electromyography sensor to measure small electrical signals generated by various muscles of medical staff 102 of body segments including, for example, knee, hip, ankle, tibialis anterior, foot, lower back, mid-back, shoulder, wrist, elbow, forearm, neck, etc.

Additional biometric sensors attached to and/or worn by medical staff 102 may include, for example, wearable sensor 312 for monitoring perspiration, which may be used, for example, to determine a stress level of medical staff 102 or an input to control air and ventilation controls in the medical suite and/or equipment worn by medical staff 102 such as personal protective equipment (PPE), heart rate monitor 318, and breathing rate monitor 320 to monitor the heart and respiration rate of medical staff 102, including heart rate variability, in order to, for example, assess potential stress and burnout of medical staff 102. An oxygenation monitor 322 may measure oxygen level of the medical staff 102 to, for example, evaluate the exertion level of medical staff 102 during certain parts of the procedure. Medical staff 102 may stand on pressure plate 306 to measure the weight distribution of medical staff 102 to, for example, provide an understanding of the balance of medical staff 102 during the medical procedure. Medical staff 102 may further wear eye motion glasses 314 to detect motion of medical staff 102's eyes and the direction of their gaze, to possibly determine, for example, mental strain, fatigue, loss of focus and concentration, and also possibly to determine if medical staff 102 needs guidance with respect to the medical procedure. In addition, medical staff 102 may wear a head-mounted sensor and/or display 316 to measure the motion and position of medical staff 102's head by way of an inertial measurement unit (IMU) or a camera detecting motion based on the position of fixed objects in the medical suite.

Sensors attached to or worn by medical staff 102 may, for example, be located on the skin of medical staff 102, over the clothing of medical staff 102, or over PPE worn by medical staff 102. Such sensors may be a wearable device such as a Zipline sensor, a Hexoskin sensor, a MotionSense sensor, a pedometer, a joint brace equipped with one or more sensors, a smartwatch including one or more sensors, etc.

Other sensors placed in the medical suite may include, for example, camera 134 that may be used for, for example, a) object detection; b) monitoring of anthropometric measurements during the medical procedure, such as, for example, distance of medical staff 102 from equipment and displays within medical suite, position of medical staff 102 while using equipment during medical procedure; c) monitoring of the distance of equipment and displays within medical suite relative to a sterile field including potential hazards within the medical suite, such as, for example smoke, fumes, dangerous light/lasers, hot or sharp surfaces, robot movements, etc., and d) monitoring of the distance between medical staff 102 to maximize efficiency. Camera 134 may employ, for example, heat sensors, motion sensors, depth sensors, etc. Camera 134 may, thus, be a component of a visual system to identify and track characteristics (i.e. measurements) of a surgeon's and/or surgical staff's posture during a procedure, possibly in combination with a central database of surgeons and surgical staff, such as ergonomic information database 106, that may include characteristics (e.g. height, age, prior injuries, etc.) of each user for customizing ergonomic alerts or alert thresholds, in combination with, possibly, details of certain wearables such as, for example, sensors 310. Measurements taken from such different types of sensors (e.g. body position/angle and pressure) may be combined when calculating ergonomic risk. The medical suite may further include a clock and/or timers to monitor the repetition, duration, and speed of specific tasks related to the medical procedure, and a thermometer and/or humidity monitor 138 to monitor the temperature and/or humidity within the medical suite to help determine and mitigate the comport and stress levels of medical staff 102.

Other sensors within the medical suite may provide additional information regarding the patient, including, for example, sensors for the patient's breathing rate and/or heart rate, skin temperature, skin moisture, pressure exerted on the patient's skin, patient movement and/or activity, the position of the patient on the bed, position and ergonomics of the patient's limbs, infrared sensors to determine patient temperature and/or movement, cameras to determine and/or record the position and/or movement of the patient, weight sensors, and accelerometers to determine patient movement, etc. Other types of patient sensors may be employed, as would be understood by one of ordinary skill.

During a medical procedure, movement of medical staff 102, the patient, medical/surgical equipment, and/or medical instruments may result in unwanted compression or pinching of a body part of the patient or medical staff 102. Additional sensors within the medical suite may be used to detect such a pinch event. Criteria for detecting a pinch event may be based on at least one sensor output indicative of at least one of position, speed, acceleration, current, power, voltage (including back EMF), and force (i.e., a load cell). In one embodiment, the criteria may include at least two of position, speed, acceleration, current, power, voltage (including back EMF), and force. In addition to or alternative to any one of these criteria, detection of a pinch event may utilize one or more external sensor outputs from at least one external sensor, such as an optical sensor (e.g., a laser or infrared sensor), a potentiometer, a gyroscope-based sensor, a magnetic sensor (e.g., a Hall effect or proximity sensor), a capacitive sensor or touch tape, and a switch (e.g., a limit switch).”

Sensors may also detect that the patient has exited the bed through the use of load cells or camera 134, or other sensors to detect the position, location, and/or movement of the patient, as discussed above. For example, sensors positioned on the patient's bed may monitor patient movement and track relative position of the patient bed relative to other components in the operating room, such as a surgical robot or medical personnel.

### Ergonomic Information Database

Ergonomic information database 106 may store information relevant to determining ergonomic “safe zones” for a medical procedure. This information may include, for example, demographic information for medical staff 102, demographic information for the patient, ergonomic standards for medical procedures, other generalized ergonomic standards, such as, for example, from human factors publications, and ergonomic data gathered during prior medical procedures. For example, ergonomics analyzer 130 may receive a particular medical practitioner's prior procedural history, including intra-operative positioning data and relative positioning of surgical apparatuses such as a surgical robot, and use this prior procedural data to determine specific ergonomic safe zones for that particular medical practitioner.

Ergonomic standards for medical procedures stored in ergonomic information database 106 may include standards for a variety of medical procedures under, for example, the strain index for hand and wrist ergonomics, which considers the intensity of exertion, duration of exertion, efforts required per minute, hand or wrist posture, speed of work, and task duration, the rapid upper limb assessment (RULA) for ergonomics of the neck, shoulder, upper arms, lower arms, hands, or wrists, which considers task repetition, posture, and force, and which may include a comparison to a recommended action level, and/or the three-dimensional static strength prediction program to determine static strength requirements for a given task, such as a sub-task within a medical procedure. Medical procedures may require use of visual displays such as computer workstations, and ergonomic standards for use of visual displays may also be included in ergonomic information database 106.

Demographic information for medical staff 102 and patients stored in ergonomic information database 106 may include, for example, height, weight, BMI, age, gender, prior injuries, pregnancy, personal general ergonomic limits, and general ergonomic limits, etc. Baseline ergonomics for medical staff 102 may be determined, for example, through completion of one or more assessments, such as the RULA employee assessment worksheet. Patient demographic information may also be considered during the medical procedure, such as to optimize positioning of medical staff 102 in order to reduce the risk of injury. For example, a patient with a heavier weight may require different position recommendations to limit ergonomic risk to medical staff 102. Also, a patient who is taller in height may require different position recommendations for various operating room apparatus, such as the patient bed or a surgical robotic arm, compared to a patient who is shorter in height.

Ergonomic data gathered during prior medical procedures and stored in ergonomic information database 106 may include, for example, recordings of all sensor data gathered during each prior medical procedure, details of all alerts and warnings issued during each prior medical procedure, baseline ergonomic data for each medical staff 102 participating in each prior medical procedure, etc.

### Ergonomics Analyzer

Prior to the medical procedure, ergonomics analyzer 130 may gather information from ergonomic information database 106 according to the type of medical procedure to be performed, the patient, and medical staff 102 who will perform the procedure, as well as anthropometric and biometric data, as discussed above. Ergonomics analyzer 130 may also receive the natural posture of medical staff 102 collected immediately prior to beginning the medical procedure. Based on this information, as well as, for example, relevant standards for ergonomics to set ergonomically safe positions, as discussed above, and the specific characteristics of medical staff 102, ergonomics analyzer 130 may calculate ergonomic “safe zones,” such as, for example, limits on a range of motion, time spent in stress positions, amount and duration of exertion, etc., as measured by the sensors discussed above. The calculated ergonomic “safe zones,” may be individualized for each medical staff 102 and may include calculation of an ergonomic baseline, possibly including an ergonomic risk baseline, for each medical staff 102 based on the information from ergonomic information database 106 as well as, for example, additional physical and ergonomic measurements of each medical staff 102 at a time near the commencement of the medical procedure.

For example, ergonomics analyzer 130 may calculate ergonomic “safe zones” for a range of motion (ROM) of a shoulder of medical staff 102 during the medical procedure to determine high, medium, or low ergonomic risk. The calculation may be based on multiple ROM criteria including, for example, a ROM zone, a percentage of time in the ROM zone, a number of repetitions, and a sustained time in a ROM position. A “high” ergonomic risk may be characterized, for example, by an elevation of the shoulder in a high ROM zone of above 60 degrees, a time in the high ROM zone of greater than or equal to 10 percent, two or more repetitions per minute of the shoulder held in the high ROM zone, and/or maintaining the shoulder in the high ROM zone for more than 30 seconds. A “medium” ergonomic risk may be characterized, for example, by an elevation of the shoulder in a medium ROM zone of between 30 and 60 degrees, a time in the medium ROM zone of greater than or equal to 20 percent without entering the high ROM zone, two or more repetitions per minute of the shoulder held in the medium ROM zone without entering the high ROM zone, and/or maintaining the shoulder in the medium ROM zone without entering the high ROM zone for more than 30 seconds. A “low” ergonomic risk may be characterized, for example, by an elevation of the shoulder in a low ROM zone below 30 degrees, a time in the low ROM zone of greater than 70 percent without entering the medium ROM zone, less than two repetitions per minute of the shoulder held in the medium ROM zone or the high ROM zone, and/or no maintaining of the shoulder in the medium ROM zone or the high ROM zone for more than 30 seconds.

During the medical procedure, ergonomics analyzer 130 may receive data from the sensors discussed above to determine the current ergonomic status of medical staff 102. Throughout the medical procedure, the current ergonomic status of medical staff 102 may be dynamically calculated and continuously updated. The determined current ergonomic status of medical staff 102 may comprise a calculated ergonomic risk score based on the determined current medical staff ergonomics and the determined one or more ergonomic safe zones. In one or more embodiments, the calculated ergonomic risk score may be calculated as a sum of multiple risk scores calculated for more than one identified posture of medical staff 102 as different tasks are performed during the medical procedure. The determined current ergonomic status of medical staff 102 may then be compared to the calculated ergonomic “safe zones” to determine the ergonomic status of medical staff 102. If any of the determined current ergonomic status of medical staff 102 exceeds the calculated ergonomic “safe zones,” or optionally, are within a predetermined threshold of the calculated ergonomic “safe zones” limits, ergonomics analyzer 130 may invoke ergonomic alert system 132 to provide feedback and alerts to medical staff 102, as discussed below. In one or more embodiments, the calculated ergonomic risk score may be compared to one or more ergonomic risk thresholds to determine appropriate feedback and alerts to provide to medical staff 102. For example, providing ergonomic feedback may include providing a first type of feedback or alert when the determined ergonomic risk score exceeds a first threshold and providing a second type of feedback or alert when the determined ergonomic risk score exceeds a second threshold. Ergonomics analyzer 130 may monitor data gathered from the sensors following the provided feedback and alerts to determine any mitigating actions taken by medical staff 102 and the effect of such actions on ergonomic status of medical staff 102.

Each medical procedure task may present a different ergonomic risk score. For example, among manual medical procedure tasks, such as orthopaedic surgery total knee replacement femoral bone cutting and femoral instrument placement may typically present high ergonomic risks, which tibial bone cutting and tibial instrument placement may present medium ergonomic risks. Among robot-assisted medical procedure tasks such as orthopaedic surgery robotic-assisted total knee replacement, for example, array placement may present a medium or high ergonomic risk and bone registration may present a medium ergonomic risk.

The analysis by ergonomics analyzer 130 may be performed based on any suitable algorithm including for example, machine learning, regression analysis, neural networks or other artificial intelligence algorithms, etc.

After the medical procedure, ergonomics analyzer 130 may update ergonomic information database 106 with information gathered during the medical procedure, as well as any ergonomic feedback or alerts generated, to be used for setting future ergonomic baseline metrics for medical procedures, report on medical procedure ergonomic metrics for each medical staff 102 to provide feedback on ergonomic risks, comparison to prior medical procedures, and/or recommendations to improve ergonomics of medical staff 102. The updated ergonomic information may also include information about the medical procedure provided by medical staff 102, for example though a questionnaire such as the Surgery Task Load Index (SURG-TLX) questionnaire.

### Application of Anthropometric and Biometric Data

As discussed above, database 106 of ergonomic information may also include anthropometric data and/or biometric data for medical staff 102.

For example, in one or more embodiments, anthropometric data may be used to determine an optimized medical suite workspace for medical staff 102, technicians, support staff, and patients by predetermining and/or adjusting medical suite workspace parameters. Such parameters may include, for example, placement, arm position(s), kinematics, etc., for surgical robot(s) 105; instrument table tray placement and size/height; patient table size, height, and placement; general workflow of the medical suite, etc. Settings of such medical suite workspace parameters may result in reduced stress on medical staff 102, technicians, support staff, and patients and/or promote physical positions and postures for medical staff 102, technicians, support staff, and patients to possibly reduce the risk of injuries and fatigue. Additional embodiments may include selection and employment of modular attachments for surgical robot(s) 105, including for example, instruments, end effectors, etc., based on the anthropometric data, and/or the provision of, for example, tooling/instruments of different lengths and/or tool/instrument handles of different sizes and shapes for medical staff 102, technicians, and support staff.

Settings and configurations of the medical suite may be saved according to, for example, type of procedure, identity of medical staff 102, technicians, and support staff, anthropometric data of medical staff 102, technicians, support staff, and patients, etc., and may be retrieved prior to a medical procedure and may be implemented, either manually or automatically, prior to the medical procedure.

In addition, in one or more embodiments, biometric data may be used to adjust medical suite workflows and spaces in response to biometric data collected for medical staff 102, technicians, and support staff. For example, biometric data may be collected for medical staff 102, technicians, and support staff and used to assess the readiness of the personnel to perform a scheduled medical procedure. Such readiness assessments may be used to schedule personnel for particular medical procedures, schedule an ordering of medical procedures, determine a number or mixture of personnel assigned to or available for a medical procedure. Biometric data may be gathered, for example, on a regular schedule, such as daily, weekly, hourly, etc., at a specified time before a medical procedure, such as an hour prior, a fixed number of hours prior, the day prior, etc. Data gathering may be performed throughout a period of time, such as a full day, daylight hours only, nighttime hours only, only during a medical procedure or other identified activity, and may be gathered continuously or at a determined frequency, such as per minute, per number of minutes, per hour, etc. Gathered biometric data may include, for example, heart rate, sleep periods, sleep quality, activity levels, weight bearing time, joint flexion, muscular contraction, etc. Additional embodiments may include incorporating biometric data for medical staff 102, technicians, and support staff into the programming and deployment of surgical robot(s) 105. For example, surgical robot(s) 105 may be programmed to provide more or less additional assistance based on the biometric data, such as providing force or positional assistance to medical staff 102, technicians, and support staff based on biometrics to support a medical procedure. Biometric data may continue to be collected during the medical procedure, and the programming of surgical robot(s) 105 may be adjusted during the procedure accordingly. That is, surgical robot(s) 105 may prove increased support during a medical procedure when for medical staff 102, technicians, or support staff are determined to have increased fatigue or stress.

### Ergonomics Alert System

As discussed above, if any of the determined current ergonomic status of medical staff 102 exceeds the calculated ergonomic “safe zones,” or optionally, are within a predetermined threshold of the calculated ergonomic “safe zones” limits, ergonomics analyzer 130 may invoke ergonomic alert system 132 to provide feedback and alerts to medical staff 102. Based on, for example, the specific ergonomic “safe zones” limit that was exceeded or approached, a relative risk associated with the ergonomic “safe zones” limit violation, in terms of both the health and potential injury to medical staff 102 and the safety of the patient, ergonomic alert system 132 may determine a type of feedback to be provided to medical staff 102 using any of the feedback and alert interfaces discussed in detail below. In one or more embodiments, a calculated ergonomic risk score for medical staff 102 may be compared to one or more ergonomic risk thresholds to determine appropriate feedback and alerts to provide to medical staff 102. That is, providing ergonomic feedback may include providing a first type of feedback or alert when the determined ergonomic risk score exceeds a first threshold and providing a second type of feedback or alert when the determined ergonomic risk score exceeds a second threshold. For example, a severe ergonomic “safe zones” limit violation, such as might lead to injury to medical staff 102 or danger to patient safety, may invoke interfaces that demand immediate attention from medical staff 102 such as, for example, lights, sounds, and vibrations. These alerts may be accompanied by text content displayed, for example, on the one or more information interfaces 124 providing more detailed information, including, for example, recommendations for adjustments or other actions to be taken by medical staff 102. On the other hand, ergonomic “safe zones” limit violations that affect the comfort of the patent or medical staff 102, or that may affect the efficiency of the medical procedure, may invoke interfaces that demand less attention from medical staff 102 such as, for example, informational messages on a display within the medical suite. Other degrees of severity of ergonomic “safe zones” limit violations may invoke more or less disruptive interfaces depending on a determined importance of mitigating the violations. In some examples, the system may automatically move a surgical robot when an ergonomic “safe zones” limit violation occurs to re-adjust the positioning of a surgical robot and/or surgical robot arm relative to one or more medical practitioners. In addition, the system may recommend changing from a manual procedure to a robot-assisted procedure or vise-versa.

The determination of the appropriate feedback and alerts provided to medical staff 102 by ergonomics analyzer 130 may be performed based on any suitable algorithm including, for example, machine learning, regression analysis, neural networks or other artificial intelligence algorithms, etc., or may be based on a correlation of ergonomic “safe zones” limit violation type and severity with a predetermined feedback and/or alert type.

### Ergonomics Analysis Operations

FIG. 4 is a flow chart of a method of optimizing medical procedure ergonomics, according to one or more embodiments. As shown in FIG. 4, in operation 405, ergonomics optimizer 110 may determine one or more ergonomics safe zones for medical staff 102 participating in the medical procedure. In operation 410, medical procedure support staff may place ergonomic sensors on medical staff 102. In operation 415, medical procedure support staff may place external sensors in the medical suite. In operation 420, medical staff 102 may begin the medical procedure. In operation 425, ergonomics optimizer 110 may monitor sensor data during medical procedure. In operation 430, ergonomics optimizer 110 may determine the ergonomics of medical staff 102 based on sensor data. In operation 435, ergonomics optimizer 110 may determine whether the measured ergonomics of medical staff 102 are outside of the determined safe zones. If the measured ergonomics of medical staff 102 are outside of the determined safe zones, then in operation 440, ergonomics optimizer 110 may display an alert to medical staff by way of ergonomic alerts and feedback 126 and the one or more information interfaces 124, and, in operation 445, ergonomics optimizer 110 may recommend correction of medical staff ergonomics to return to the determined safe zone. Ergonomics optimizer 110 may then continue with operation 465. If the measured ergonomics of medical staff 102 are outside of the determined safe zones, then in operation 455, ergonomics optimizer 110 may display, to medical staff 102 by way of ergonomic alerts and feedback 126 and the one or more information interfaces 124, an indication that medical staff ergonomics are in determined safe zone. Ergonomics optimizer 110 may then continue with operation 465. In operation 465, ergonomics optimizer 110 may provide recommendations for adjustments of medical staff 102, medical equipment such as a patient bed or surgical robot, and/or the patient to optimize the ergonomics of medical staff 102. In operation 470, medical staff 102 may complete the medical procedure. In operation 475, ergonomics optimizer 110 may update ergonomic information database 106, including the ergonomic baselines of medical staff 102, based on sensor data recorded during medical procedure. In operation 480, ergonomics optimizer 110 may display a report of the ergonomics of medical staff 102 during medical procedure.

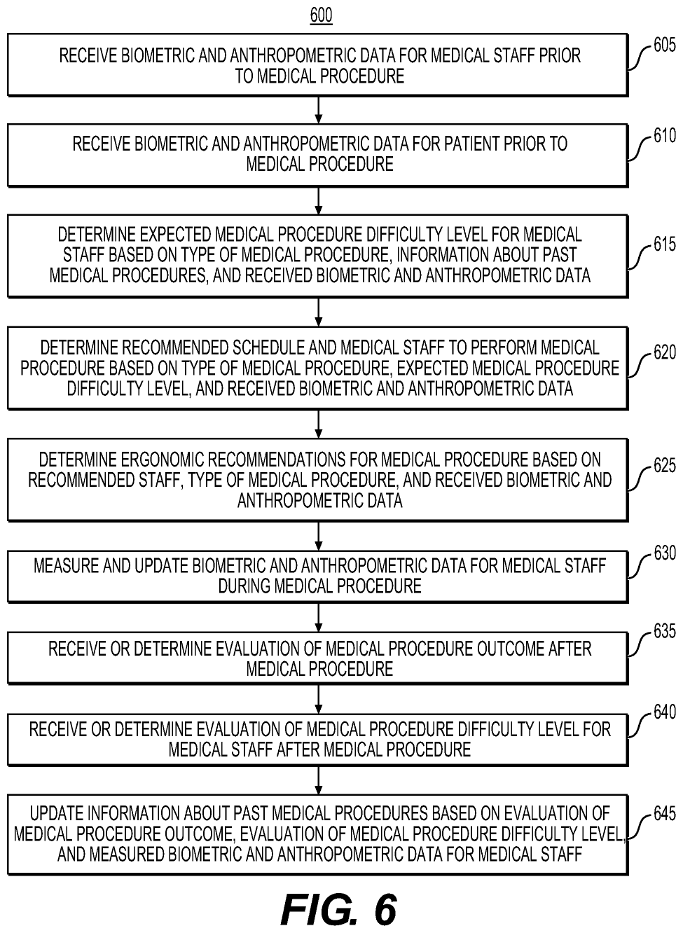

FIG. 6 is a flowchart of a method of optimizing medical procedure ergonomics, according to one or more embodiments. As shown in FIG. 6, in operation 605, ergonomics optimizer 110 may receive biometric and anthropometric data for medical staff 102, technicians, and support staff prior to a medical procedure. The medical staff 102, technicians, and support staff for whom biometric and anthropometric data are received in operation 605 may include medical staff 102, technicians, and support staff who are not eventually scheduled to perform or participate in the medical procedure. In operation 610, ergonomics optimizer 110 may receive biometric and anthropometric data for the patient prior to the medical procedure. In operation 615, ergonomics optimizer 110 may determine an expected medical procedure difficulty level for medical staff 102, technicians, and support staff based on, for example, the type of medical procedure, information about past medical procedures, such as may be stored in database 106 of ergonomic information, and the received biometric and anthropometric data. In operation 620, ergonomics optimizer 110 may determine a recommended schedule for the medical procedure and recommended medical staff to perform the medical procedure based on, for example, the type of medical procedure, the expected medical procedure difficulty level, and the received biometric and anthropometric data. In operation 625, ergonomics optimizer 110 may determine one or more ergonomic recommendations for the medical procedure, such as are discussed in detail above, based on the recommended staff, the type of medical procedure, and the received biometric and anthropometric data. In operation 630, ergonomics optimizer 110 may measure and update biometric and anthropometric data for medical staff 102, technicians, and support staff during medical procedure. In operation 635, ergonomics optimizer 110 may receive or determine an evaluation of the medical procedure outcome after the medical procedure. In operation 640, ergonomics optimizer 110 may receive or determine an evaluation of the medical procedure difficulty level for medical staff 102, technicians, and support staff after the medical procedure. In operation 645, ergonomics optimizer 110 may update information about past medical procedures, such as may be stored in database 106 of ergonomic information, based on the evaluation of medical procedure outcome, the evaluation of medical procedure difficulty level, and the measured biometric and anthropometric data for medical staff 102, technicians, and support staff.

### Ergonomics Feedback and Alerts

As discussed above, during the medical procedure, ergonomics optimizer 110 may monitor ergonomic data relating to medical staff 102 to determine, by way of ergonomics analyzer 130, whether the ergonomics remain in a determined ergonomic “safe zone.” Ergonomics optimizer 110 may then, possibly by way of ergonomic alert system 132 provide feedback and alerts 126 to medical staff 102. Feedback and alerts 126 by way of one or more information interfaces 124 may be positioned within the medical suite and may take the form of, for example, lights/sounds/vibrations/equipment controls 510 and/or a visual feedback display 510. Visual feedback display 510 may display, for example, graphical alerts be accompanied by text content displayed, for example, on the one or more information interfaces 124 providing more detailed information, including, for example, recommendations for adjustments or other actions to be taken by medical staff 102. Lights/sounds/vibrations/equipment controls 510 may include, for example, lights on surgical robot 105 or other medical equipment or elsewhere within the medical suite. The lights may be visible only to one or more medical staff 102, such as through a head-mounted display or other augmented reality application. Lights/sounds/vibrations/equipment controls 510 may include, for example, warning sounds or spoken alerts or feedback within the medical suite. The sounds may be audible only to one or more medical staff 102, such as through a headset. Lights/sounds/vibrations/equipment controls 510 may include, for example, vibrations or other haptic feedback provided by way of, for example, pressure pad 306, motion wearable sensors 310, one or more medical tools or instruments, etc. For example, a wearable sensor 310 at the lower back of medical staff 102 may alert medical staff 102 to adjust their posture to relieve lower back strain, or a medical tool or instrument may vibrate to indicate that greater or lesser force is required or that a different tool may be required. Lights/sounds/vibrations/equipment controls 510 may include, for example, an adjustment to a table height, movement of a position of medical robot 105, adjustment of a support for a portion of medical staff 102's body, or adjustment of the patient's position, etc. Medical staff posture information and other ergonomic information may be used to optimize the position of, for example, robot 105 and the patient which improve the ergonomics of medical staff 102. Information interfaces 124 that may be positioned within the medical suite may further provide visual and/or textual feedback information 520. Visual/textual feedback 520 may include, for example, an overview of medical staff 102's body 530 with associated alert symbols 540 indicating areas that may have exceeded ergonomic “safe zones.” Alert symbols 540 may be differentiated, for example, by differing colors, symbols, text, patterns, or animations to indicate differing levels of severity. Visual/textual feedback 520 may further include textual descriptions 545 of each alert, such as, for example a description of excessive neck flexion or lower back strain. Visual/textual feedback 520 may also include recommendations 550 for adjustments to medical staff 102 ergonomics to optimize performance of the medical procedure. Other information provided by visual/textual feedback 520 may include an indication to medical staff 102 to increase or decrease the pace of the medical procedure, an indication of a sequence of steps in the medical procedure to minimize strain on medical staff 102, a display of a recommended adjustment of a positioning of a surgical robotic arm, and/or an indication of a best position of medical staff 102, including a posture of the individual medical staff 102, and position of medical staff 102's head/neck/limbs/hands/feet, as well as a best position of medical staff 102 relative to medical equipment, the patient, and other medical staff strain and injury risk. Visual/textual feedback 520 may also include, for example, a warning if an ergonomic limit is being approached or exceeded, or an unadvised motion of a person is detected, such that an aspect of the medical procedure will deviate from an ergonomic “safe zone,” an indication if medical staff 102 violates a sterile field, or an indication that a number of medical staff 102 in the medical suite is above a threshold leading to higher chance of infection due to sterile field violation.

As discussed above, one or more embodiments may include determining ergonomic safe zones based on one or more ergonomic standards, data from prior medical procedures, and information about a particular medical procedure to be performed. The analysis may further be based on past history of medical staff 102, stored and measured ergonomic data of medical staff 102, and demographic information of medical staff 102. Thus, the ergonomic safe zones, thresholds for generating feedback and alerts, and the content of the generated feedback and alerts may be personalized to the individual, such as, for example, by incorporating demographics, the prior health and injury history of medical staff 102, and the current ergonomic status of medical staff 102. Thus, the disclosed one or more embodiments may provide more accurate ergonomic analysis overall for each individual medical staff 102.

Furthermore, one or more embodiments may employ a broad variety of sensors, including particular wearable sensors to gather ergonomic information of medical staff 102 that go beyond just imaging and analysis on the medical staff 102 activity within the medical suite. Thus, the disclosed one or more embodiments may provide richer and more accurate ergonomic analysis overall.

Any suitable system infrastructure may be put into place to allow user control of an interactive audiovisual environment, and engagement assessment. FIGS. 2, 3, and 5, and the preceding discussion, provide a brief, general description of a suitable computing environment in which the present disclosure may be implemented. In one embodiment, any of the disclosed systems, methods, and/or graphical user interfaces may be executed by or implemented by a computing system consistent with or similar to that depicted in FIGS. 2, 3, and 5. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain functions, are described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), and/or the Internet. Similarly, techniques presented herein as involving multiple devices may be implemented in a single device. In a distributed computing environment, program modules may be located in both local and/or remote memory storage devices.

Aspects of the present disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A computer-implemented method for optimizing medical procedure ergonomics, the method comprising:
receive baseline ergonomic data from a database;
determine one or more ergonomic safe zones based on the receive baseline ergonomic data;
receive ergonomic measurements from sensors within a medical suite and sensors attached to a body of a medical staff;
determine current medical staff ergonomics based on the received baseline ergonomic data and received ergonomic measurements during a medical procedure;
determine whether the determined current medical staff ergonomics exceed any of the determined one or more ergonomic safe zones; and
upon determining that the determined current medical staff ergonomics exceed any of the determined one or more ergonomic safe zones, providing ergonomic feedback to the medical staff.

2. The computer-implemented method of claim 1, wherein the current medical staff ergonomics are dynamically calculated and continuously updated while the medical staff is performing the medical procedure.

3. The computer-implemented method of claim 1, wherein the ergonomic feedback is one or more of a light, a sound, a vibration, and a visual display with text content.

4. The computer-implemented method of claim 1, wherein the sensors within the medical suite and the sensors attached to the body of the medical staff include one or more of a motion wearable sensor, eye motion glasses, a head mounted display, a biometric sensor, a pressure sensor, a camera, a clock, a timer, a thermometer, a humidity sensor, and a position sensor attached to a robotic surgical instrument.

5. The computer-implemented method of claim 1, further comprising:
calculating an ergonomic risk score for the medical staff based on the determined current medical staff ergonomics and the determined one or more ergonomic safe zones.

6. The computer-implemented method of claim 5 wherein providing ergonomic feedback comprises providing a first visual alert when the determined ergonomic risk score exceeds a first threshold and providing a second visual alert when the determined ergonomic risk score exceeds a second threshold.

7. The computer-implemented method of claim 1, further comprising:
calculating an ergonomic risk baseline for the medical staff based on personal characteristics of the medical staff stored in the database.

8. A system for optimizing medical procedure ergonomics, the system comprising:
a data storage device storing instructions for optimizing medical procedure ergonomics in an electronic storage medium; and
a processor configured to execute the instructions to perform a method including:
receive baseline ergonomic data from a database;
determine one or more ergonomic safe zones based on the receive baseline ergonomic data;
receive ergonomic measurements from sensors within a medical suite and sensors attached to a body of a medical staff;
determine current medical staff ergonomics based on the received baseline ergonomic data and received ergonomic measurements during a medical procedure;
determine whether the determined current medical staff ergonomics exceed any of the determined one or more ergonomic safe zones; and
upon determining that the determined current medical staff ergonomics exceed any of the determined one or more ergonomic safe zones, providing ergonomic feedback to the medical staff.

9. The system of claim 8, wherein the current medical staff ergonomics are dynamically calculated and continuously updated while the medical staff is performing the medical procedure.

10. The system of claim 8, wherein the ergonomic feedback is one or more of a light, a sound, a vibration, and a visual display with text content.

11. The system of claim 8, wherein the sensors within the medical suite and the sensors attached to the body of the medical staff include one or more of a motion wearable sensor, eye motion glasses, a head mounted display, a biometric sensor, a pressure sensor, a camera, a clock, a timer, a thermometer, a humidity sensor, and a position sensor attached to a robotic surgical instrument.

12. The system of claim 8, wherein the system is further configured for:
calculating an ergonomic risk score for the medical staff based on the determined current medical staff ergonomics and the determined one or more ergonomic safe zones.

13. The system of claim 12, wherein providing ergonomic feedback comprises providing a first visual alert when the determined ergonomic risk score exceeds a first threshold and providing a second visual alert when the determined ergonomic risk score exceeds a second threshold.

14. A non-transitory machine-readable medium storing instructions that, when executed by a computing system, causes the computing system to perform a method for optimizing medical procedure ergonomics, the method including:
receive baseline ergonomic data from a database;
determine one or more ergonomic safe zones based on the receive baseline ergonomic data;
receive ergonomic measurements from sensors within a medical suite and sensors attached to a body of a medical staff;
determine current medical staff ergonomics based on the received baseline ergonomic data and received ergonomic measurements during a medical procedure;
determine whether the determined current medical staff ergonomics exceed any of the determined one or more ergonomic safe zones; and
upon determining that the determined current medical staff ergonomics exceed any of the determined one or more ergonomic safe zones, providing ergonomic feedback to the medical staff.

15. The non-transitory machine-readable medium of claim 14, wherein the current medical staff ergonomics are dynamically calculated and continuously updated while the medical staff is performing the medical procedure.

16. The non-transitory machine-readable medium of claim 14, wherein the ergonomic feedback is one or more of a light, a sound, a vibration, and a visual display with text content.

17. The non-transitory machine-readable medium of claim 14, wherein the sensors within the medical suite and the sensors attached to the body of the medical staff include one or more of a motion wearable sensor, eye motion glasses, a head mounted display, a biometric sensor, a pressure sensor, a camera, a clock, a timer, a thermometer, a humidity sensor, and a position sensor attached to a robotic surgical instrument.

18. The non-transitory machine-readable medium of claim 14, the method further comprising:
calculating an ergonomic risk score for the medical staff based on the determined current medical staff ergonomics and the determined one or more ergonomic safe zones.

19. The non-transitory machine-readable medium of claim 18, providing ergonomic feedback comprises providing a first visual alert when the determined ergonomic risk score exceeds a first threshold and providing a second visual alert when the determined ergonomic risk score exceeds a second threshold.

20. The non-transitory machine-readable medium of claim 14, the method further comprising:
calculating an ergonomic risk baseline for the medical staff based on personal characteristics of the medical staff stored in the database.
